(19) United States
(12) Patent Application Publication
(10) Pub. No.: US 2011/0276036 A1
(43) Pub. Date: Nov. 10, 2011

(54) COUPLING FOR TUBULAR ELEMENTS

(75) Inventors: Martin SPRANGER, Hechingen (DE); Thilo JOOST, Hechingen (DE)

(73) Assignee: NOVALUNG GMBH

(21) Appl. No.: 13/104,348

(22) Filed: May 10, 2011

Related U.S. Application Data
(63) Continuation of application No. 11/732,615, filed on Apr. 3, 2007.

(30) Foreign Application Priority Data
Apr. 3, 2006 (DE) 10 2006 016 211.0

(51) Int. Cl. A61M 25/16

(52) U.S. Cl. 604/535

(57) ABSTRACT

The invention relates to a coupling for tubular elements, with at least one plug and one socket, which plug can be inserted into the socket and can be locked therein, and both the plug and the socket have, extending approximately along their longitudinal axis, a channel for a fluid, said channel being arranged in such a way that the fluid can flow through the coupling in a locked state thereof. The plug can be locked in the socket in at least two locking positions, and the socket and/or the plug has at least one opening such that, in a first locking position, the opening connects the channel for a fluid to the environment, and, in a second locking position, the channel for a fluid is isolated from the environment. The invention relates further to a cannula that comprises such a coupling.

### CROSS REFERENCE TO RELATED APPLICATIONS

This application claims priority under 35 U.S.C. §119(d) from German patent application No. 10 2006 016 211.0 filed Apr. 3, 2006. The content of the above patent application is incorporated by reference herein in its entirety.

### FIELD OF THE INVENTION

The present invention relates to a coupling for tubular elements, with at least one plug and one socket, which plug can be inserted into the socket and can be locked therein, and both the plug and the socket have, extending approximately along their longitudinal axis, a channel for a fluid, said channel being arranged in such a way that the fluid can flow through the coupling in a locked state thereof.

The invention further relates to a cannula that comprises such a coupling.

For coupling tubular elements, it was for a long time customary to use short tube sections whose external diameter was slightly greater than the internal diameter of the tubular elements that are to be coupled. The tubular elements to be connected were simply pushed onto the coupling, the difference in diameter ensuring the fixed connection. If appropriate, corrugations were formed on the coupling in order to achieve a still more secure connection.

A connection of this kind has the disadvantage that considerable forces are often needed to push the tubular elements onto the coupling, which is often undesirable, especially in the field of medicine, because the forces applied may be transmitted to a patient and cause injuries.

### RELATED PRIOR ART

To solve this problem, a two-part coupling, disclosed for example in U.S. Pat. No. 5,052,725, is composed of a plug and of a socket, and the tubular elements that are to be connected can each be connected in a separate state to the plug and to the socket, respectively, and they can then be connected to one another without excessive force being applied.

However, it has been found that undesired accumulation of air may take place in such couplings. Moreover, it is often desirable, especially in the field of medicine, to be able to introduce other substances into the fluid transported into the tubular elements, or to be able to remove samples from this fluid.

In the known couplings, such removal of samples or introduction of other substances is possible only by undoing the connection, carrying out the desired procedure, and then restoring the connection. The removal of undesired air can also be done only by separating and then restoring the connection. This is complicated and means that the flow of fluid through the coupling often has to be interrupted.

An object of the invention is therefore to describe a coupling for tubular elements from which air can be easily removed and in which, even during operation, substances can be introduced from outside into the flow of fluid, or samples can be re-moved from the flow of fluid.

### SUMMARY OF THE INVENTION

According to the invention, this object is achieved by the fact that the plug can be locked in the socket in at least two locking positions, and by the fact that the socket and/or the plug has at least one opening such that, in a first locking position, the opening connects the channel for a fluid to the environment, and, in a second locking position, the channel for a fluid is isolated from the environment.

A tubular element in accordance with the invention is understood as an elongate structure that comprises, approximately along its longitudinal axis, at least one channel for transport of a fluid. Such tubular elements include, for example, rigid tubes and also flexible hoses.

The locking can be effected in any manner known to a person skilled in the art, provided that it allows a secure, but releasable connection to be established. Examples of devices for this locking include grooves in combination with locking lugs or spring-mounted balls, or closures of the bayonet type.

By provision of the abovementioned opening, it is now possible to connect the plug and the socket of the coupling to one another such that, in a first locking position, an access route from the outside to the internal channel for a fluid is still present, allowing air to be removed from the coupling or allowing substances to be removed from the flow of fluid or introduced into it. In a second locking position, the opening is closed, and the channel is completely cut off from the environment.

In this way, for example, air can be removed from the coupling without said coupling having to be undone, and without the flow of fluid through the coupling having to be interrupted. Since the plug remains connected to the socket during this period, the access to the flow of fluid can also be made more quickly. By virtue of the plug and the socket remaining connected to one another, the opening can be opened and closed much more easily and with just one hand.

It will be understood here that the expressions “first locking position” and “second locking position” are used only to distinguish the locking positions from one another and do not imply anything about their sequence. It is entirely conceivable that, when the plug is inserted into the socket, the second locking position will be reached before the first locking position.

In one embodiment of the invention, it is preferable if an actuating element is present with which the locked engagement of the plug and socket can be cancelled.

Although it is possible to establish a locked engagement that can be can-celled simply by pulling on the plug and socket, a relatively large force is needed in order to cancel a sufficiently secure connection of this kind, which force may, for example, be transmitted to a patient and cause injuries to the latter. By the provision of an actuating element, the locked engagement can be cancelled without excessive forces being applied to the coupling. The plug and the socket can thus be moved from one locking position to the other or separated from one another without any particular force being needed, with the result that this can be done using just one hand. The type of actuating element depends on the type of locking mechanism and can, for example, comprise a rotary ring for a bayonet-type lock, or a push-button if the locked engagement is obtained via locking lugs that engage in grooves.

In another embodiment of the invention, it is preferred if the plug has at least two annular grooves on an outer circumferential surface, and the socket has at least one locking lug that engages in one of the annular grooves in the locking positions.

Such a construction has proven particularly simple both in terms of manufacture and operation, and, at the same time, it results in a sufficiently secure locking between the plug and the socket. The provision of annular grooves also allows the plug and socket to be turned relative to one another in the locked state.

In one embodiment of the aforementioned measure, the locking lug is formed by a slotted ring which is arranged in an annular groove in an inner circumferential surface of the socket and protrudes above the inner circumferential surface at least at one location, in which case the actuating element has in particular a wedge with which the slotted ring can be spread in such a way that an engagement of the ring in one of the annular grooves of the plug can be cancelled.

According to this embodiment, the locking lugs can, for example, be formed by two sides of a ring that protrude above the inner circumferential surface of the socket and engage in the annular grooves of the plug in the locking positions. If a wedge is now inserted into the slot of the ring, the two protruding sides of the ring are pressed outwards and no longer engage in the grooves. The locked engagement is thus cancelled.

In this way, the socket can be produced in one piece except for the ring. The ring can be based, for example, on a normal slotted lock washer, which makes this construction simple and inexpensive to produce. In addition, by using a ring, the locking can take place over a relatively large part of the circumferential face of the plug, which makes the connection particular secure.

In another embodiment of the invention, it is preferred if the opening has a connector piece on the outer face, in particular a socket for a syringe.

This embodiment has proven advantageous particularly when the coupling is used in the field of medicine, since in this way samples can easily be removed from the flow of fluid, or substances can be introduced into the flow of fluid with a syringe. Moreover, such a connector piece can also be used to remove air in a controlled manner, thus providing an indication of whether there are any leaks in the system.

In another embodiment of the invention, the plug and the socket are made of plastic.

In this way, a coupling is created that is easy and inexpensive to produce and is also light. Moreover, plug and coupling can be designed as a disposable article, which is advantageous particularly in the medical sector, since in this way the coupling no longer has to be resterilized.

In another embodiment of the invention, the plug has a seal in the front area.

This measure ensures a particularly leaktight connection.

In another embodiment, the opening is arranged in the socket.

The effect of this measure is that, even in the locking position in which the channel is isolated from the environment, the opening can, for example, be connected to a syringe, such that a sample can be removed, without excessive escape of liquid.

It will be appreciated that the aforementioned features and the features still to be explained below can be used not only in the respectively cited combination but also in other combinations or singly, without departing from the scope of the present invention.

### BRIEF DESCRIPTION OF THE DRAWINGS

The invention is now explained in more detail with reference to the attached drawings, in which:

FIG. 1 shows a plug and a socket in cross section, in the unlocked state,

FIG. 2 shows a plug and a socket in cross section, in a first locking position,

FIG. 3 shows a plug and a socket in cross section, in a second locking position, and

FIG. 4 shows a perspective view of a cannula with a coupling.

### DETAILED DESCRIPTION OF PREFERRED EMBODIMENTS

In FIG. 1, a coupling is designated in its entirety by reference number 10.

The coupling 10 comprises a plug 12 and a socket 14. Along its longitudinal axis, the plug 12 has a channel 16 for a fluid, and the socket 14, along its longitudinal axis, has a corresponding channel 18. Both the plug 12 and the socket 14 are in this case made of plastic.

A first annular groove 20 and a second annular groove 22 are formed in an outer circumferential surface of the plug 12.

An annular groove 24 is formed in an inner circumferential surface of the socket 14. A slotted ring 26 with a triangular cross section is arranged in this annular groove 24. The ring 26 is arranged in the annular groove 24 in such a way that at least part of it protrudes above the inner circumferential surface of the socket 14.

The socket 14 also has an opening 28, which is here designed as a simple bore through which the channel 18 is connected to the environment.

The plug 12 is now coupled to the socket 14 by inserting the plug 12 into the socket 14 in the direction of an arrow 30.

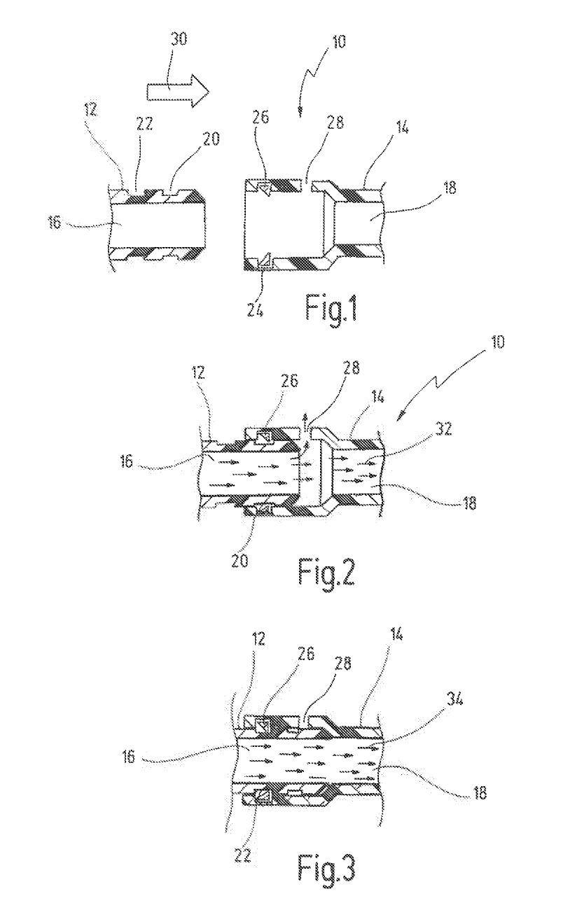

In FIG. 2, the coupling 10 is shown in a first locking position after the movement indicated by the arrow 30 in FIG. 1. It will be seen from this view that the ring 26 now engages in the first annular groove 20 of the plug 12. In this way, the plug 12 and the socket 14 are connected to one another by being firmly locked.

The channel 18 is here still connected to the environment via the opening 28. If a fluid, in this case a patient's blood that is being conveyed into an extracorporeal circuit, now flows through the channel 16, 18, as indicated by the arrows 32, it can emerge through the opening 28 into the environment. If air has now accumulated at the coupling 10, it is forced outwards through the opening 28 by the fluid stream 32, and the coupling 10 is thus vented.

FIG. 3 shows the situation after a further movement, as indicated by the arrow 30 in FIG. 1. Here, the plug 12 has been inserted further into the socket 14. The ring 26 now comes to lie in the second annular groove 22 and locks the plug 12 in this position in the socket 14.

It will also be clear from this view that the opening 28 is now blocked by the plug 12, such that the channel formed by the channels 16 and 18 is completely isolated from the environment, and only a flow of fluid through the channels 16 and 18 is now possible, as indicated by the arrows 34.

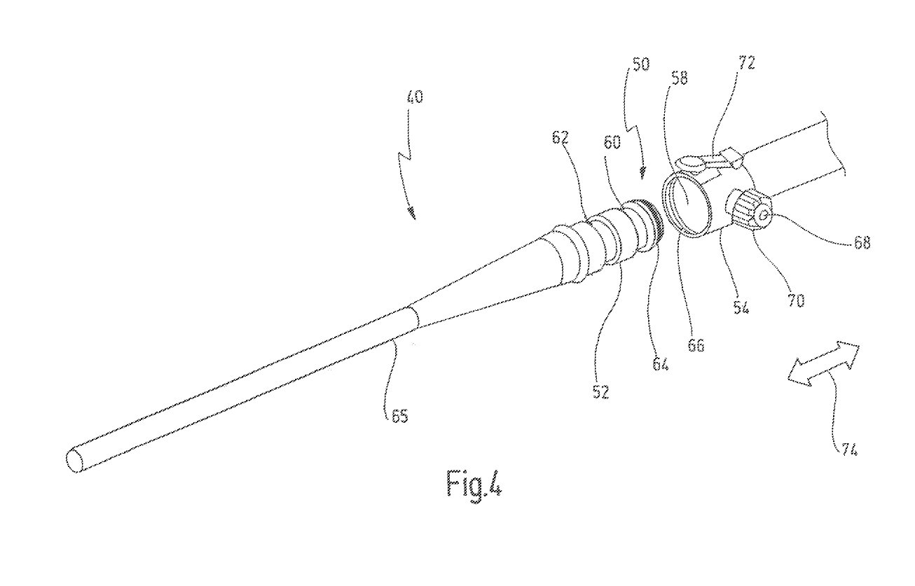

In FIG. 4, a cannula is designated in its entirety by reference number 40.

This cannula 40 has a coupling 50 composed of a plug 52 and of a socket 54. A channel 58 serving for the passage of a fluid can be seen in the socket 54.

Two annular grooves 60 and 62 are formed in an outer circumferential surface of the plug 52, and a seal 64 is additionally arranged in the front area of the plug 52. On the side facing away from the seal 64, the plug is adjoined by the actual cannula 65, which is here designed as a seamless cannula. This cannula 65 is composed of a wire-reinforced tube, which is fitted onto a plastic cone, the wire reinforcement being arranged between an inner layer and an outer layer of the tube.

In the inner circumferential surface of the socket 54, there is a slotted ring 66 which is used to lock the plug 52 in the socket 54. Arranged on the side of the socket 54 there is an opening 68, which here has a connector piece 70 in the form of a socket for a syringe. The channel 58 of the socket 54 is connected to the environment by way of this opening 68.

The socket 54 moreover comprises an actuating element 72, which is de-signed here as a push-button. With this push-button, a wedge can be pressed into a slot present in the ring 66, as a result of which the ring 66 is spread open. The spreading of the ring 66 means that the locked engagement of the plug 52 in the socket 54 can be cancelled, and the two parts can be separated from one another.

During use, for example as a cannula for conveying blood into an extra-corporeal blood circuit, the cannula 65 is introduced into a vessel with the aid of a dilator. The dilator can, for example, be connected securely to the cannula 65 via the annular groove 60.

After the cannula has been fitted in place, the dilator can be removed, and the socket 54, which is connected to a tube as its end remote from the cannula 65, is moved towards the left in the direction of a double arrow 74 and fitted onto the plug 52. A syringe (not shown here) is arranged on the connector piece 70. The socket 54 is now moved towards the left in the direction of the double arrow 74 until the ring 66 comes to lie in the first annular groove 60 of the plug 52. In this state, the channel 58 of the socket 54 is still connected to the environment, in this case to the syringe, via the opening 68.

By drawing up the syringe, an operator can now check whether there are air bubbles present at the coupling site, or he can use the syringe to remove a sample of blood via the connector piece 70. When the operator has satisfied himself that the coupling is as it should be, he moves the socket 54 further in the direction of the double arrow 74 towards the left, as a result of which the ring 66 now comes to lie in the second annular groove 62 and the plug 52 closes the opening 68 from the inside, such that the inner channel is now completely isolated from the environment.

If an operator now wishes, for example, to inject a medicament into the blood circuit, he can place it in a syringe and insert this syringe into the connector piece 70. He then actuates the actuating element 72, such that the locked engagement of the plug 52 in the socket 54 is cancelled. He now moves the socket 54 towards the right in the direction of the double arrow 74, specifically to such an extent that the ring 66 now comes to lie again in the first annular groove 60 of the plug 52. In this way, the channel 58 is now once again connected to the environment via the opening 68, and the desired medicament can now be injected via the syringe into the blood stream.

After the injection procedure is complete, the operator again moves the socket 54 towards the left in the direction of the double arrow 74, as a result of which the opening 68 is closed by the plug 52.

After completion of the procedure, or when the cannula is to be connected to another tube, an operator once again actuates the actuating element 72, as a result of which the ring 66 is spread open and no longer engages in the annular groove 62 of the plug 52. The operator now moves the socket 54 towards the right in the direction of the double arrow 74 and separates it completely from the plug 52, without any particular forces being exerted on the patient.

The plug 52 can now be coupled to another tubular element, or the cannula 65 can be removed from the patient.

1-11. (canceled)

12. A cannula comprising a coupling having a plug and a socket.
